(19) United States
(12) Patent Application Publication
(10) Pub. No.: US 2014/0214095 A1
(43) Pub. Date: Jul. 31, 2014

(54) SYSTEMS AND DEVICES FOR THE REDUCTION AND ASSOCIATION OF BONES

(71) Applicant: The Trustees of Columbia University in the City of New York, New York, NY (US)

(72) Inventors: Melvin P. Rosenwasser, Palisades, NY (US); Eugene Jang, College Point, NY (US)

(73) Assignee: The Trustees of Columbia University in the City of New York, New York, NY (US)

(21) Appl. No.: 14/229,412

(22) Filed: Mar. 28, 2014

Related U.S. Application Data
(63) Continuation of application No. PCT/US12/58038, filed on Sep. 28, 2012.
(60) Provisional application No. 61/563,324, filed on Nov. 23, 2011.
(60) Provisional application No. 61/541,898, filed on Sep. 30, 2011.

(51) Int. Cl. A61B 17/68; A61B 17/86; A61B 17/88

(52) U.S. Cl. CPC A61B 17/68; A61B 17/8866; A61B 17/86
USPC 606/301; 606/86 R

(57) ABSTRACT

In accordance with the disclosed subject matter, a medical device is provided which comprises a body having a first end and a second end, a first engaging member positioned adjacent the first end of the body and adapted to operatively engage a bone, and a second engaging member positioned adjacent the first end of the body and adapted to operatively engage the bone, wherein the position of at least one engaging member is adjustable with respect to the body.

### CROSS REFERENCE

This application claims priority to U.S. Provisional Patent Application Ser. No. 61/563,324, filed Nov. 23, 2011, and U.S. Provisional Patent Application Ser. No. 61/541,898, filed Sep. 30, 2011, the disclosures of each are hereby incorporated by reference in their entireties.

### BACKGROUND OF THE DISCLOSED SUBJECT MATTER

1. Field

The disclosed subject matter relates to systems and apparatus for medical procedures, e.g., the reduction and association of bones. More particularly, the disclosed subject matter is directed to devices that facilitate the reduction and association of the scaphoid and Innate bones.

2. Background

“Reduction” is a medical procedure that restores a bone fracture or dislocation to its correct alignment. Generally, when a bone fractures, the fragments typically lose their alignment and become displaced or angulated. In order for the fractured bone to heal without any deformity the bone fragments must be re-aligned to their normal anatomical position. Orthopedic surgeons attempt to recreate the normal anatomy of the fractured bone by reduction. The reduced bone fragments are maintained in proper alignment by an implant. The accuracy of the reduction can be verified by x-ray. Reduction may also refer to the re-alignment of bones to their normal anatomical position after ligaments connecting two or more bones become disrupted, either as a result of a traumatic injury or over time due to normal wear and tear.

Reduction techniques can be closed or open. In a closed reduction the fractured bone pieces are aligned into their correct positions manually and without making incisions. Occasionally, medical instruments are used to provide a fraction force to help separate the bone fragments so that they can be easily adjusted. In an open reduction procedure, an incision is made in the skin and the broken bone is viewed. Then the bone fragments are brought together and typically fixed together with an implant, such as screws and pins.

An example of an open reduction procedure involves the reduction and association of the scaphoid and lunate bones in the wrist. A procedure sometimes referred to as “RASL.” Typically, the RASL procedure is a treatment for scapholunate dissociation or subacute static scapholunate instability.

Scapholunate dissociation or subacute static scapholunate instability is the most common type of carpal instability. It is generally caused by the scapholunate interosseus ligament (FIG. 1; 1006) breaking down, and results in the scaphoid (1002) and lunate (1004) bones separating and rotating out of alignment. Left untreated, the instability can lead to severe wrist disability and arthritis associated with scapholunate advanced collapse.

Prior art methods and medical tools for treating scapholunate dissociation have drawbacks. They limit post-operative wrist motion and often prevent subsequent salvage procedures. More recently, the RASL procedure has been found to provide safe and effective treatment for chronic static scapholunate dissociation by re-aligning the scaphoid and lunate bones, restoring function, and reducing pain. Currently, surgeons performing the RASL procedure simultaneously use 1.6 mm-thick metal Kirschner wires (“K-wires”) to manipulate the bones, a headless cannulated screw to maintain the positioning of the bones post-operatively, and a guide wire to position the screw at the site.

A major difficulty in treating scapholunate dissociation is that there is very little clearance within the bones afforded by currently available medical tools used to perform in the procedure (e.g., K-wires, bone clamps, etc), a large number of bones at the site, and a compact area within which to perform the procedure. To wit, there is very little clearance and visibility between the K-wires for the guide wire and the screw, making it difficult and error-prone to properly manipulate the bones using K-wires while leaving enough room for the guide wire and screw to be introduced.

Currently, there are no medical tools or instrumentation available for precisely performing reduction and association techniques on bones, and in particular anatomical sites that have small or compact bones such as in the wrist. The only tools available to surgeons for performing open reduction procedures including RASL are non-specific, generic clamps, and K-wires. Such tools are sub-optimal and provide no repeatable way to ensure that the screw is implanted in the proper axis. The success of the procedure often depends on the surgeons' experience in making educated guesses based on anatomical and biomechanical landmarks and skill in positioning or repositioning the guide wire based on radiographic images. The success is further complicated by the K-wires employed to hold the bones in place getting in the way of the smaller guide wire, sometimes causing deflection or inhibition. As the guide wire is typically 1.0 mm in diameter and the K-wire is typically about 1.6 mm in diameter, the guide wire is often deflected upon contact with the thicker and stronger K-wire. Referring to FIG. 2 a fluoroscopic image from a RASL procedure demonstrates deviation of the guidewire as a result of collision with the thicker K-wire.

Identification of the proper position for the guide wire and drilling a pilot hole (FIG. 3; 3000) for the cannulated screw 3002 thereover is also difficult and often requires a very skilled surgeon. Ideal placement of the screw 3002 is along the axis representing the instantaneous center of motion between the scaphoid 1002 and lunate 1004 bones in the wrist. Usually, the axis is parallel to the radial inclination and coincident with the mid-waist of the scaphoid and the apex of the lunate. Years of experience are typically required to find the correct axis. Currently, a jig is used to facilitate the identification of the correct axis, such as the jig disclosed in U.S. Pat. No. 5,312,412 to Whipple. However, the jig is not designed for RASL procedures, and does not perform well in simplifying the identification of the proper axis, and, as such, is rarely used in such procedures.

The screw 3002 used in maintaining reduced bones post-operatively also has drawbacks. The smooth shank allows rotation about the axis without sacrificing tensional stability, but the implant cannot accommodate movements of the joint in any plane other than rotation strictly about the axis of the implant. Accordingly, toggle is usually not possible, and physiologic motion is curtailed. Moreover, the axis of the screw must align precisely with the instant center of motion of the joined bones to avoid stressing both the screw and bone. Such stresses may lead to excessive loosening of the screw, restriction of motion, and pain. Furthermore, damage to the bone may lead to irreversible damage to the bone, and breakage of the screw due to excessive bending moments is fairly common.

There still remains an unmet need for improved medical systems and devices to reduce and associate bones, and in particular, reducing and associating the scaphoid and lunate bones. Effective mechanical replacements for ligaments have thus not found widespread use, either in the wrist or in any other joints of the body. There also remains an unmet need to facilitate guide wire positioning and pilot hole drilling for implant placement in the proper physiological axis. There also remains an unmet need to provide an implant that can accommodate bending to maximize the amount of physiologic motion between reduced and associated bones post-operatively. The disclosed subject matter meets these needs.

### SUMMARY OF THE DISCLOSED SUBJECT MATTER

In accordance with the disclosed subject matter, a medical device is provided which comprises a body having a first end and a second end, a first engaging member positioned adjacent the first end of the body and adapted to operatively engage a bone, and a second engaging member positioned adjacent the first end of the body and adapted to operatively engage the bone, wherein the position of at least one engaging member is adjustable with respect to the body. In some embodiments, at least one of the first engaging member and the second engaging member includes at least two bone-contacting points. In some embodiments, the first engaging member includes at least two bone-contacting points and the second engaging member includes at least two bone-contacting points. In some embodiments, the first engaging member includes a curved toothed bone-contacting surface and the second engaging member includes a single bone-contacting point. In still other embodiments, the first engaging member includes a curved toothed bone-contacting surface and the second engaging member includes a curved toothed bone-contacting surface.

The disclosed subject matter also includes a medical apparatus, comprising a body, a barrel having a first portion adapted to engage a first bone wherein the first portion includes an angled tip to fit a step-off angle of the second bone, and a targeting member having a second portion adapted to engage the bone, wherein the distance or spacing between the first portion and second portion is adjustable. In some embodiments, the barrel is hollow, and/or rotatable. In still other embodiments, the bone-engaging portion includes at least two bone-contacting points to confer stability on the contact point with the bone.

The disclosed subject matter also includes a medical implant comprising a longitudinal body having a first end portion, a second end portion, and an intermediate portion wherein the intermediate portion is bendable. In some embodiments, the first end portion and the second end portion are rigid. In other embodiments, the intermediate portion is made of Nitinol. In still other embodiments, the implant is made of a nickel titanium alloy wherein the concentration of nickel is greatest in the intermediate portion and least at the end portions. In still other embodiments, the intermediate proportion is composed of a mesh-like structure to allow greater bendabilty or flexibility. In other embodiment, the intermediate portion of the body is cut such as by laser to increase the flexibility of the section and render it bendable.

In another aspect, a medical tool for reducing first and second bones is provided. The tool includes first, second, and third arms. The first and second arms are adapted to receive first and second medical devices as described above. The third arm is a dial up member that can measure the angle or rotation necessary to properly re-align bones. The medical tool provides a user with a method to facilitate the proper re-alignment of rotated bones. The dial up member can be used to correctly position the first and second medical devices so that the bones are gripped and moved or rotated to their proper positions.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

### BRIEF DESCRIPTION OF THE DRAWINGS

FIG. 1 is a schematic representation of structures within the human hand;

FIG. 2 is a fluoroscopic image from an RASL procedure demonstrating deviation of the guidewire as a result of collision with the thicker K-wire;

FIG. 3 is a schematic representation of scaphoid lunate fixation with a cannulated screw;

FIGS. 4A and 4B are schematic representations of the incisions made during an RASL procedure;

FIGS. 4C and 4D are schematic representations of bone manipulation using K-wires

FIG. 5 is a schematic representation of bone burring in order to induce a biological healing response;

FIG. 6 is a schematic representation of the prior art method of clamped K-wires being used as a crude method to hold the reduction;

FIG. 7 is a fluoroscopic image from an RASL procedure illustrating the minuscule clearances facing surgeons during the procedure;

FIG. 8 is a schematic representation of a system for performing a medical procedure in accordance with the disclosed subject matter;

FIG. 9A to 9C are schematic representations of the medical device in accordance with the disclosed subject matter;

FIGS. 10A-D are schematic representations of embodiments of the medical device in accordance with the disclosed subject matter;

FIG. 11 are photographs of various clamping devices;

FIGS. 12A-C are schematic representations of embodiments of the medical device in accordance with the disclosed subject matter;

FIG. 13A-C are schematic representations of the medical apparatus in accordance with the disclosed subject matter;

FIG. 14 is a schematic representation of the combination article in accordance with the disclosed subject matter;

FIG. 15A-B are schematic representations of the medical implant in accordance with the disclosed subject matter;

FIG. 16A-C are schematic representations of an embodiment of the medical implant;

FIG. 17A-C are schematic representations of an embodiment of the medical implant;

FIGS. 18A and 18B are schematic representations of an embodiment of the medical implant;

FIG. 19A-C are schematic representations of an embodiment of the medical implant;

FIG. 20 is a schematic representation of scapholunate fixation with a cannulated screw;

FIG. 21A is a schematic representation of scaphoid lunate fixation with a cannulated screw, demonstrating the allowed axis of motion with use of current implants.

FIG. 21B is a schematic representation of scaphoid lunate fixation with a cannulated screw, illustrating the toggle (rotation in other planes) that is currently impossible with existing technology; and

FIG. 22 is a schematic representation of the dial up reduction tool in accordance with the disclosed subject matter.

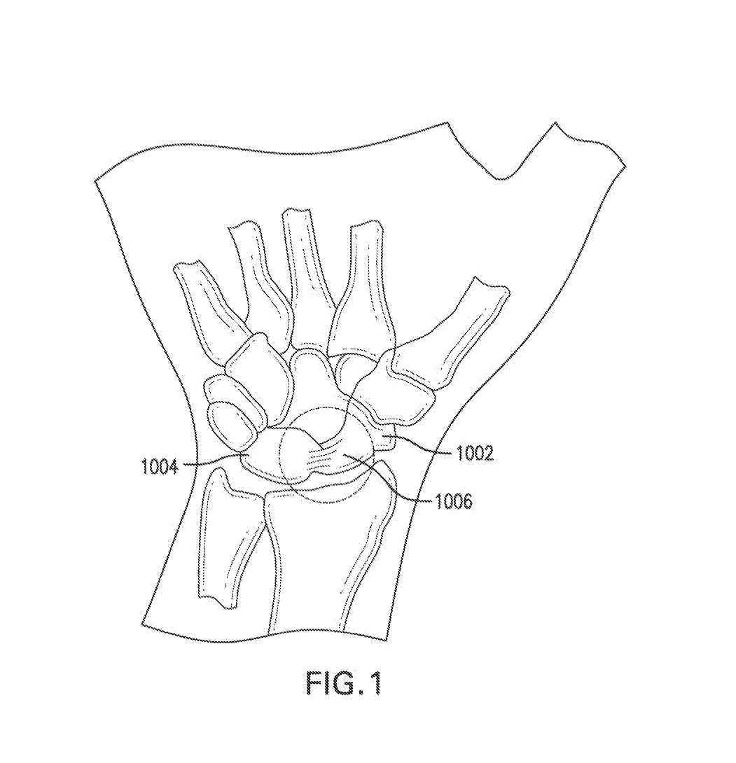

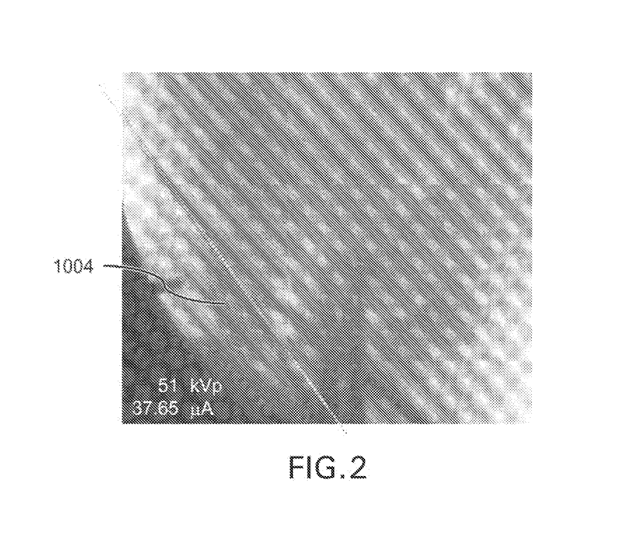

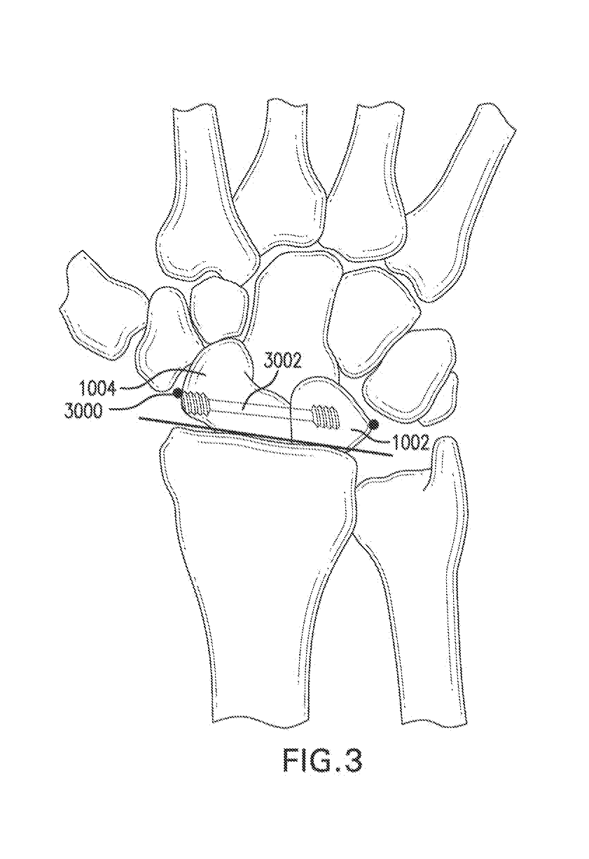

### DETAILED DESCRIPTION OF THE EXEMPLARY EMBODIMENTS

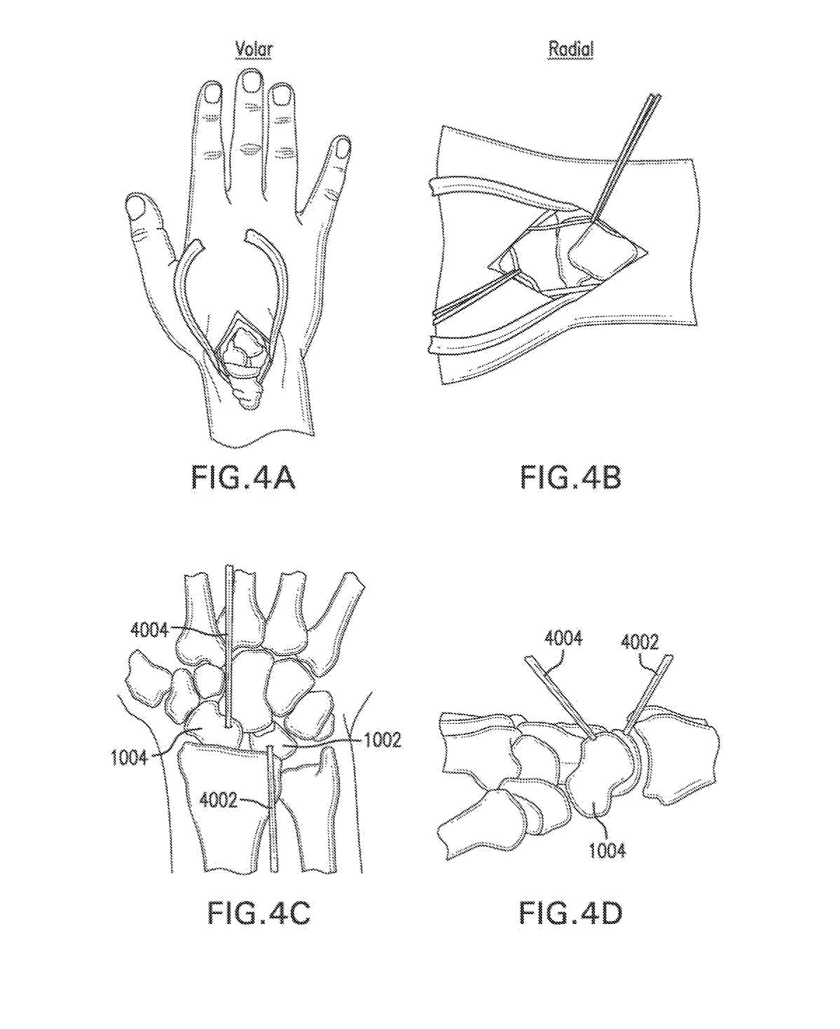

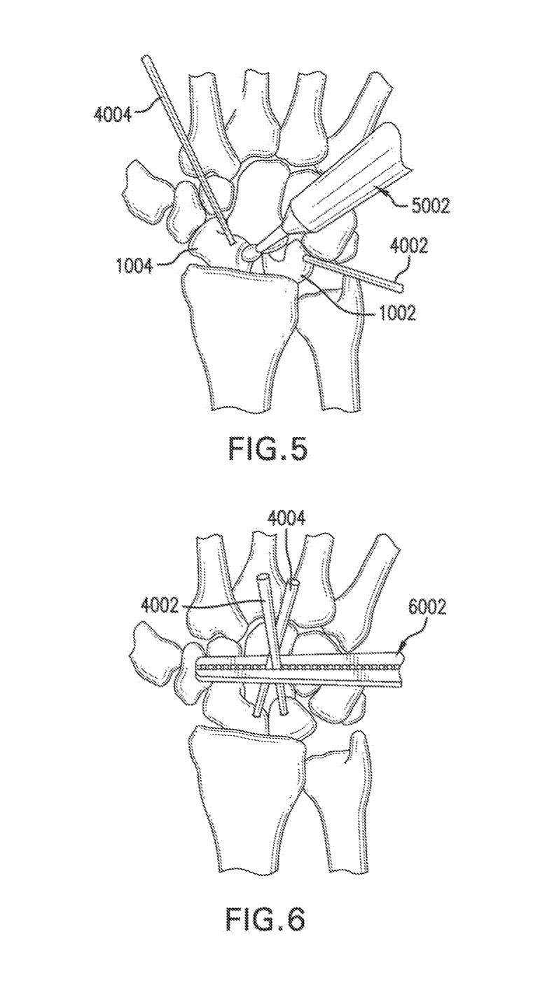

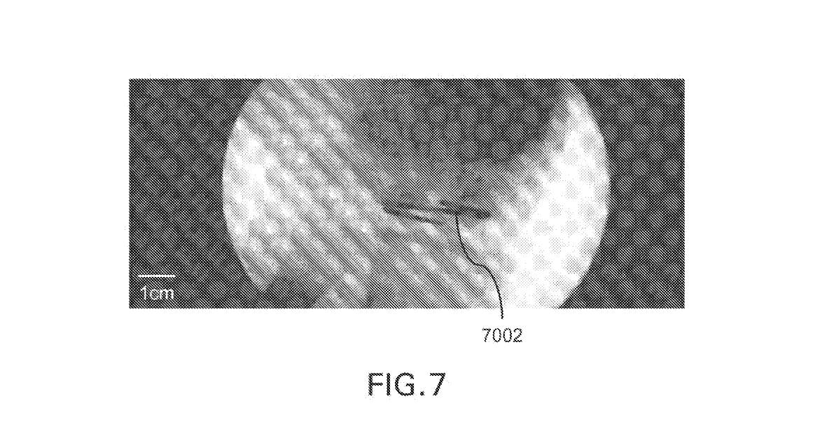

During a RASL procedure, a volar incision is made exposing the scaphoid, lunate and capitate bones, and a radial incision is made exposing the radial sensory nerve, radial artery scaphoid and radial styloid, as shown in FIGS. 4A and 4B. In prior art techniques, K-wires (FIG. 4C; 4002, 4004) are driven into both the scaphoid 1002 and lunate 1004 bones. The K-wires 4002, 4004 are then manipulated to align the scaphoid and lunate bones, as shown in FIG. 4D. The K-wires are placed in such a way that the scaphoid can be rotated backwards and the lunate can be rotated forwards to correct the rotational deformity. The inner chondral surfaces of the two bones 1004, 1002 are typically burred (FIG. 5; 5002) to induce a healing response that allows for the formation of a soft tissue connection between the two bones. The K-wires 4002, 4004 are then rotated (causing rotation of the bone) to the correct position and the K-wires 4002, 4004 are held together using a Kocher clamp (FIG. 6; 6002). With the two bones 1002, 1004 held in place, the radial incision is used to first take off the radial styloid. Then, a guide wire is inserted along the axis of the instant center of motion of the two bones. The position of the guide wire is confirmed using fluoroscopic imaging. Once confirmed, a pilot hole is made using a cannulated drill. Finally, the scaphoid and lunate bones are de-rotated and a headless cannulated screw, e.g., Herbert-Whipple screw, (FIG. 7; 7002) is implanted to associate the bones (1004, 1006) and hold them in place. The cannulated screw is typically formed from hollow titanium and includes two sets of threads of varying pitches with a smooth shank therebetween. The smooth shank of the screw allows for relative motion between the two bones (1002, 1004), and the varying thread pitches apply compressive forces on the bones to maintain the reduction of fractures post-operatively.

The devices, systems and methods presented and claimed herein provide improved medical instrumentation and methods for reducing and associating bones in general and in particular, the scaphoid and innate bones.

Although the disclosed subject matter is suited for manipulation of the carpal bones, e.g., rotating and reducing the scaphoid and lunate bones and associating them to each other, it will become apparent from the description below that the disclosed subject matter is useful for the reduction and association of other bones. Accordingly, although reference to the exemplary embodiments that follow are described in the context of RASL procedures, and the scaphoid and lunate bones, the devices, system, and methods described and claimed can be utilized for the reduction and association of other bones and joints.

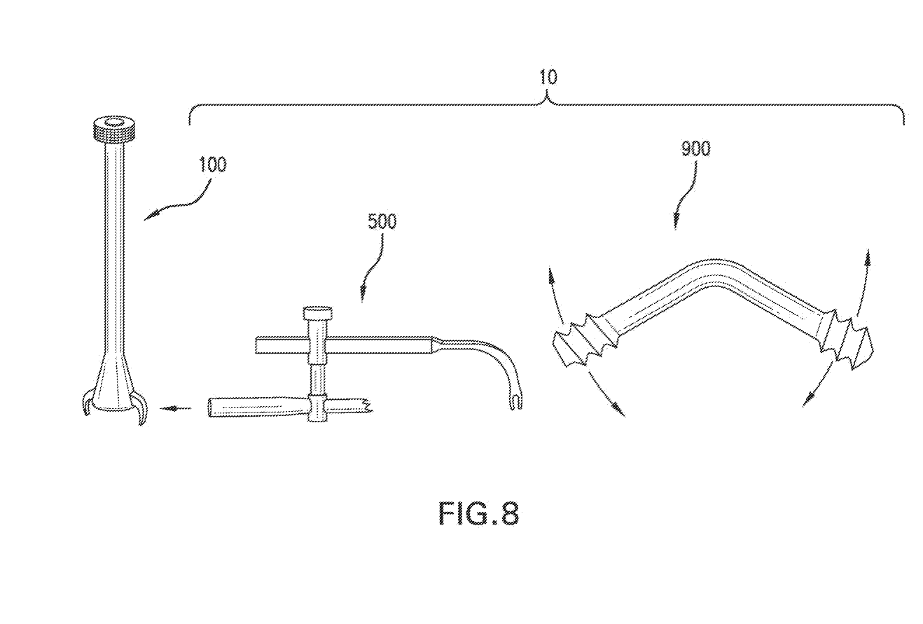

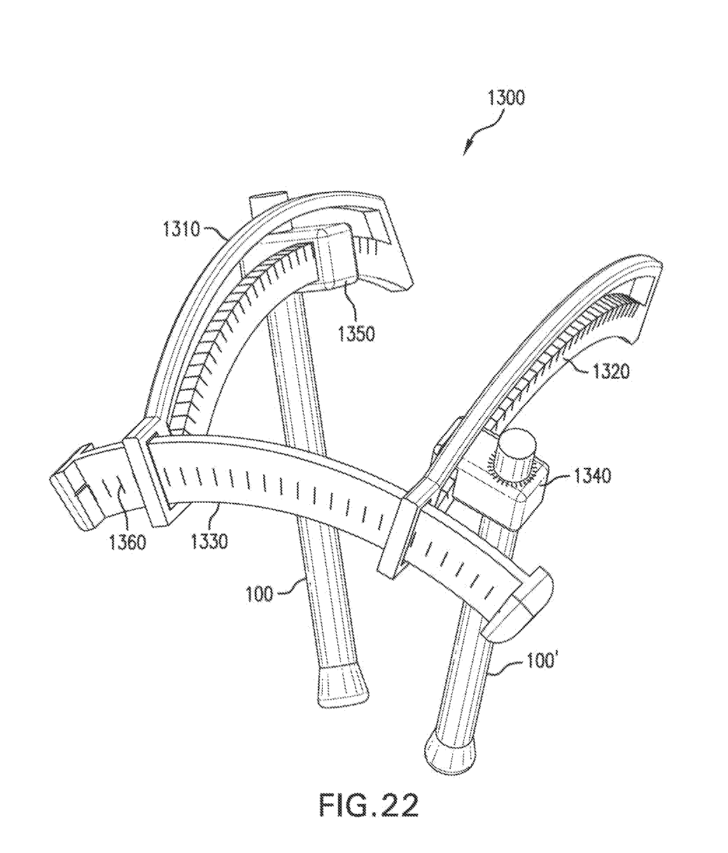

An exemplary embodiment of a system in accordance with the disclosed subject matter is shown in FIG. 8 and is designated generally by reference character 10. As shown in FIG. 8, the system 10 generally includes a medical device 100 configured to clamp and grip bones in need of reduction, a medical apparatus 500 to aid the proper positioning of an implant for maintaining the alignment of the reduced bones, and a medical implant 900 for maintaining the bones in proper alignment post-operatively. In some procedures, two medical devices 100 can be used, for example, to grip or clamp two different bones. For example, one medical device 100 can be used to engage the scaphoid bone and a second medical device 100 can engage the lunate bone. The system may further include a reduction tool to aid in obtaining and maintaining a precise reduction in preparation for introduction of an implant, as shown in FIG. 22 and described below.

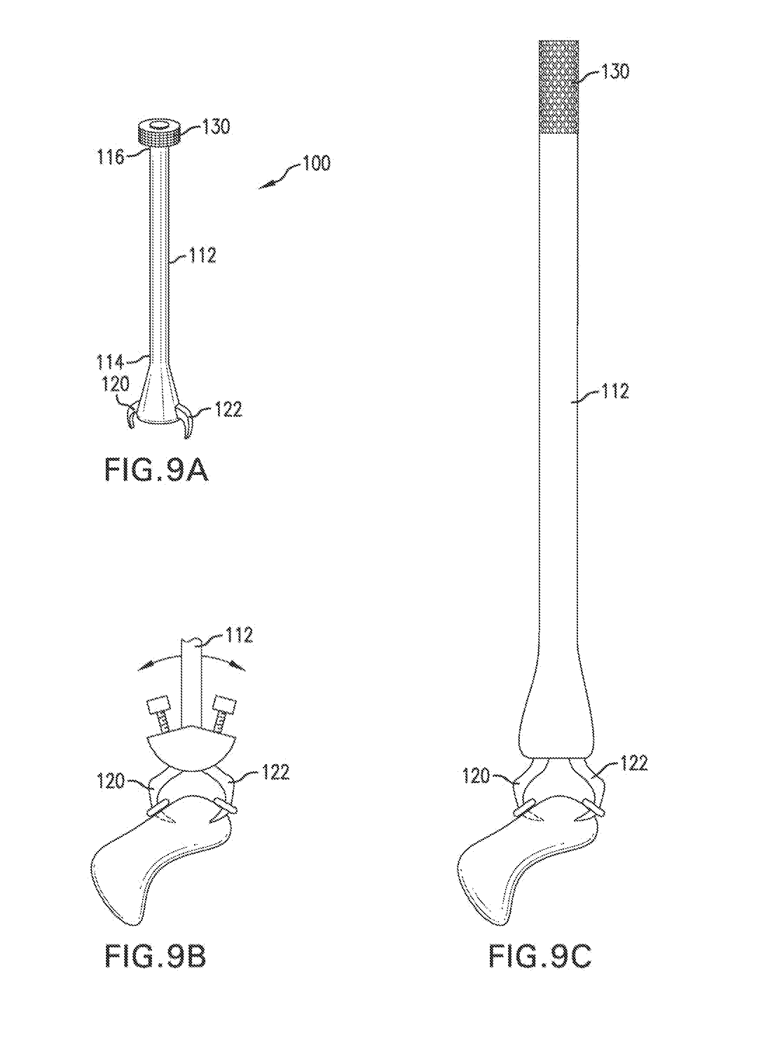

In one embodiment, medical device 100 is designed to clamp or grasp bone, and in particular, bones that are, for example, small and/or curved, e.g., engage a carpal bone, for example, the scaphoid bone and/or lunate bone. Referring to FIG. 9A, the medical device 100 can include a tubular body 112 having a first end 114 and a second end 116, a first engaging member 120, a second engaging member 122, and a controller 130, such as a knob. The engaging members are located near the first end 114 and the knob is located near the second end 116 of the medical device 100. The engaging members are adjustable between a first position, in which the first and second engaging members 120, 122 are spaced apart, and a second position, in which the first and second engaging members 120, 122 are closer together to grip a bone, as shown in FIGS. 9B and 9C. The range of motion for each engaging member 120,122, for example, can range from a 90 degree angle relative to body 112 to a 0 degree angle as the engaging members approach one another.

The movement of the engaging members from the first to second positions can be actuated by controller 130 that is operatively connected to a mechanism to translate movement of the controller to movement of the engaging members. The movement can be designed such that the engaging members incrementally move from the first to second positions. Controller 130, e.g., twisting knob, can be actuated by a mechanism enclosed within the body 112 of medical device 100 to effect adjustment or movement of the engaging members 120, 122 with respect to the body 112 individually or simultaneously. In this manner, the engaging members 120, 122 can move from the first position to the second position to secure the medical device 100 to bone. For example, in some embodiments, one engagement member is adjustable and one engagement member is fixed. In other embodiments, both engagement members 120, 122 are adjustable with respect to the body 112.

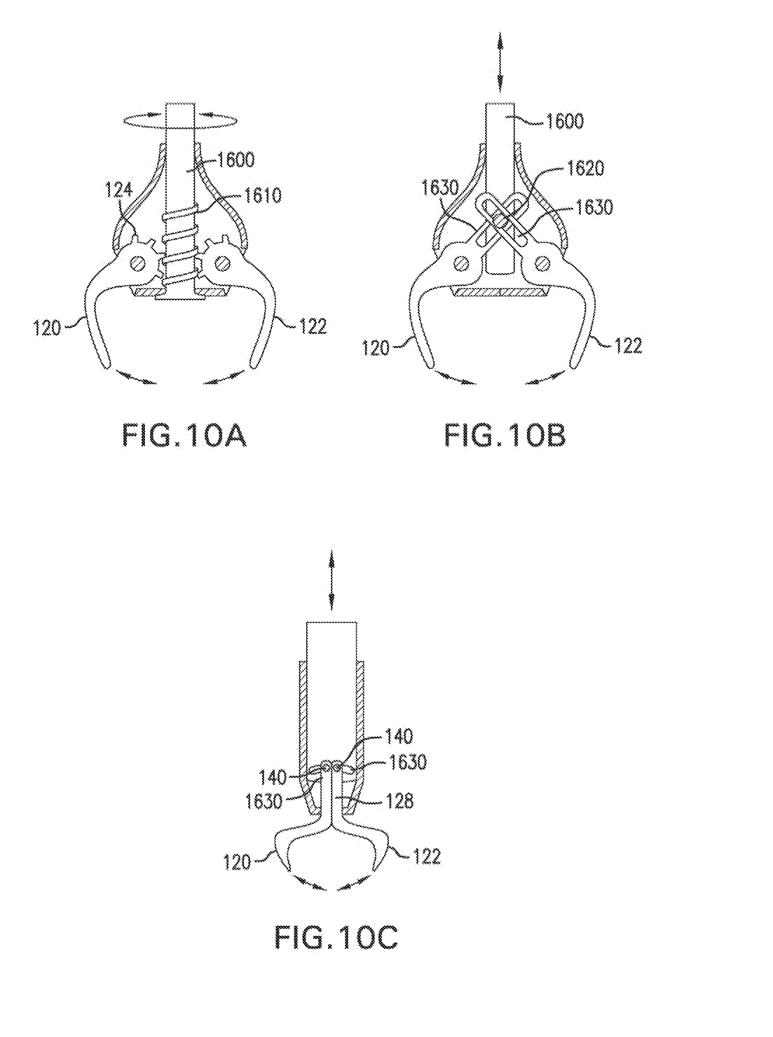

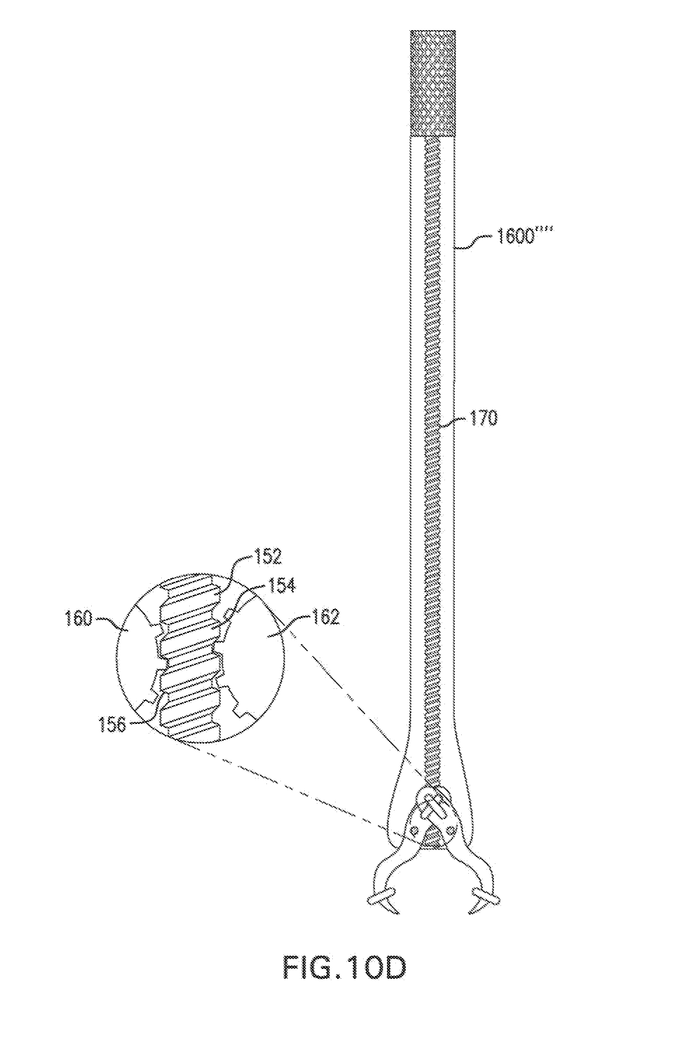

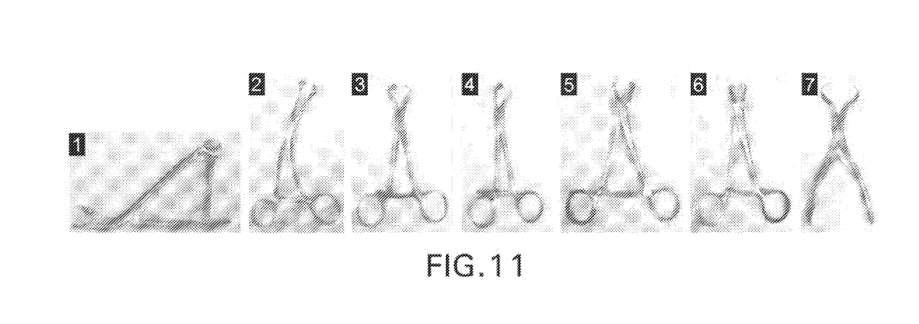

In some embodiments, the controller 130 is a knurled knob. However, other types of controllers can be employed, such as a button, lever, and the like. As stated above, the controller is operatively connected to a mechanism to translate movement of the controller to movement of the engaging members. Referring to FIGS. 10A, 10B, 10C, and 10D various force transmission mechanisms can be implemented in medical device 100 body 112 to actuate movement of the engaging members 120, 122 by controller 130.

In one embodiment, a lead screw or “corkscrew” mechanism can be employed as illustrated in FIG. 10A. In accordance with this embodiment, shaft 1600 includes a plurality of threads 1610 along a length thereofngaging members 120, 122 further includes one or more gear teeth 124 configured to engage one or more of the plurality of threads 1610. Shaft 1600 is operatively engaged to controller 130 of the medical device 100 such that rotation of the controller 130 translates to rotation of the shaft 1600. As the threads rotate with the shaft 1600, the engagement with the gear teeth 124 cause the engaging members 120 and 122 to move from a first position to a second position.

In another embodiment, as depicted in FIG. 10B, a “grasper” mechanism can be employed. In this embodiment, mechanism includes shaft 1600′ that includes one or more rivets 1620 on a surface thereof. Engaging members 120, 122 can further include one or more slots 126 configured to engage the one or more of the rivets 1620. Shaft 1600′ is operatively engaged to controller 130 of the medical device 100 such that actuation of the controller 130 translates to linear movement of the shaft 1600′. As the one or more rivets 1620 linearly move while engaged to the one or more slots the engaging members 120 and 122 move from a first position to a second position.

In yet another embodiment, a “jeweler's pickup” mechanism can be used, as illustrated in FIG. 10C. In this embodiment, mechanism includes shaft 1600″ that includes one or more slots 1630 on a surface thereofngaging members 120, 122 can further include one or more arms 128 including one or more rivets 140 configured to engage the one or more of the slots 1630 of shaft 1600″. Shaft 1600′ is operatively engaged to controller 130 of the medical device 100 such that actuation of the controller 130 translates to linear movement of the shaft 1600″. The linear movement of the shaft 1600″ and one or more slots 1630 engaged to the rivets 128 causes the engaging members 120 and 122 move from a first position to a second position.

In yet another embodiment, the body 112 of medical device 100 can include within shaft 1600″″ a longitudinal member 150 having a plurality of threads (152, 154, 156) along its length. Engaging members 120 and 122 can include first and second gears 160, 162 having a plurality of teeth 164, 166, 168 engaged to the plurality of threads 152, 154, 156. The linear movement of the longitudinal member 150 and plurality of threads engaged to gear teeth causes the engaging members 120 and 122 to move from a first position to a second position. It will be understood, however, that other mechanisms can be employed to translate movement of the controller to movement of the engaging members 120, 122, such as for example a rack and pinion arrangement and the like.

During use, medical device 100 is placed in proximity to bone to be gripped with the engaging members in the first open position. When twisted or otherwise actuated, the controller causes the engaging members to move from a first position to a second position to securely engage the bone, e.g., clamp the bone. The medical device 100 provides the ability to rotate and reduce the bone without the use of any K-wires. Accordingly, no K-wires are required to perform a bone reduction using the medical device 100 described herein. Thus, the site of the procedure can remain uncluttered and visible to the operator or surgeon, and leave the full interior of the bones accessible for the implant, unlike prior art methods and tools.

The medical device 100 can be configured to fit the anatomy of the bones to be reduced, e.g., scaphoid and lunate bones. Currently, the only available instruments available to clamp bones are generic clamps, which are non-specific and unsuitable for carpal bone anatomy. It has been found that generic clamps are ill-suited for open procedures, especially for the wrist or other small bones. Generic, “all-purpose,” bone clamps are often too bulky and difficult to use to rotate and move bones, making them unsuitable for fine movements in a small space such as those encountered in wrist surgery. They are also not well-designed for bones with a curved surface or within sites where there is very close tolerances.

Referring back to FIG. 9A, when the engaging members 120, 122 are in the second, e.g., substantially closed, position, medical device 100 grips the carpal bones with sufficient force such that the bones can be reduced in the flexion/extension plane, without slipping in the pronation/supination or radial/ulnar deviation planes. Unlike K-wires, which must penetrate deep into the carpal bones to permit proper reduction, medical device 100 grips the surface of the bone, which results in minimal damage to the bone. In some embodiments, the engaging members penetrate the bone surface, such as for example, about 1 to 5 mm of penetration depending on how many points or teeth engage the bone, as illustrated in FIGS. 9B and 9C. In some embodiments, the engaging members do not penetrate the bone surface. The greater the number of points or teeth 140, 144 the greater the distribution of force on the bone. For example, increasing from two teeth to four teeth, (or more) increases the frictional force while the bone is grasped by medical device 100. This increase in frictional force is sufficient to secure and engage the bone. For example and not limitation, the medical device 100 can provide sufficient penetration through the cartilage surrounding the bone while not penetrating the bone.

In various embodiments, the engaging members 120, 122 of medical device 100 may have different bone-contacting configurations. Cadaveric testing was undertaken using seven existing instruments, including Ulrich™ Bone Holding Forceps with Speed Lock, Curved Forceps w/Open Circle Ends, Tiemann™ Clamp (one sharp tip, one platform tip), Tiemann™ Clamp (extra sharp), Finger Clamp, Finger Clamp (sharper, shinier), and a Double Action Clamp, as shown in FIG. 11. Two independent observers conducted the tests and provided feedback to determine desirable bone-contacting configurations. The tests were performed on a cadaver scaphoid and lunate and found to be within two standard deviations of the mean scaphoid and lunate morphology in each mode of variation. User feedback was collected on a Pugh chart, shown in Table 1. Table 1 evidences that none of the generic bone clamps currently available are well-suited for complex anatomy, such as wrist anatomy.

TABLE 1
RASL Functional Surface Pugh Chart

| | | Grip Strength | Stability | | | | | | |
|---|---|---|---|---|---|---|---|---|---|
| | | (Strong Hold | (Doesn't Slip | Fit with | Fit with | Ease of Use/ | Visibility | | |
| | | During Derotation | in P/S or R/U | Anatomy | Anatomy | Surgeon | of | Bone | |
| UID | Brief Description of Tool | in F/E Plane | Planes) | (Scaphoid) | (Lunate) | Comfort | Anatomy | Damage | Overall |
| 1 | Ulrich Bone Holding Forceps with | 2 | 1 | 1 | −2 | 2 | −1 | 0 | 21.5 |
| | Speed Lock | 2 | 1 | 1 | 1 | 2 | −1 | −1 | |
| 2 | Curved Forceps w/Open Circle | 1 | 1 | 1 | −1 | 1 | −2 | 1 | 0.5 |
| | Ends | −1 | 0 | −1 | −1 | 0 | −1 | 2 | |
| 3 | Tiemann Clamp (1 Sharp tip, 1 | 3 | 0 | 0 | 0 | 0 | 0 | −1 | 2 |
| | Platform Tip) | 1 | 0 | 0 | 0 | 0 | 0 | −1 | |
| 4 | Tiemann Clamp (Extra Sharp) | 3 | 0 | 0 | 0 | 0 | 0 | 0 | 0 |
| | | 3 | 0 | 0 | 0 | 0 | 0 | 0 | |
| 5 | Finger Clamp | 2 | 2 | 0 | −2 | 1 | −1 | −2 | 10.5 |
| | | 2 | 1 | 1 | 1 | 1 | 0 | −2 | |
| 6 | Finger Clamp (Sharper, Shinier) | 2 | 1 | 0 | −2 | 1 | −1 | −1 | 20.5 |
| | | 2 | 2 | 1 | 1 | 1 | 0 | −1 | |
| 7 | Double Action Clamp | 1 | −1 | 2 | 1 | 0 | 1 | −1 | 9.5 |
| | | 1 | 1 | 1 | 1 | 1 | −1 | 1 | |

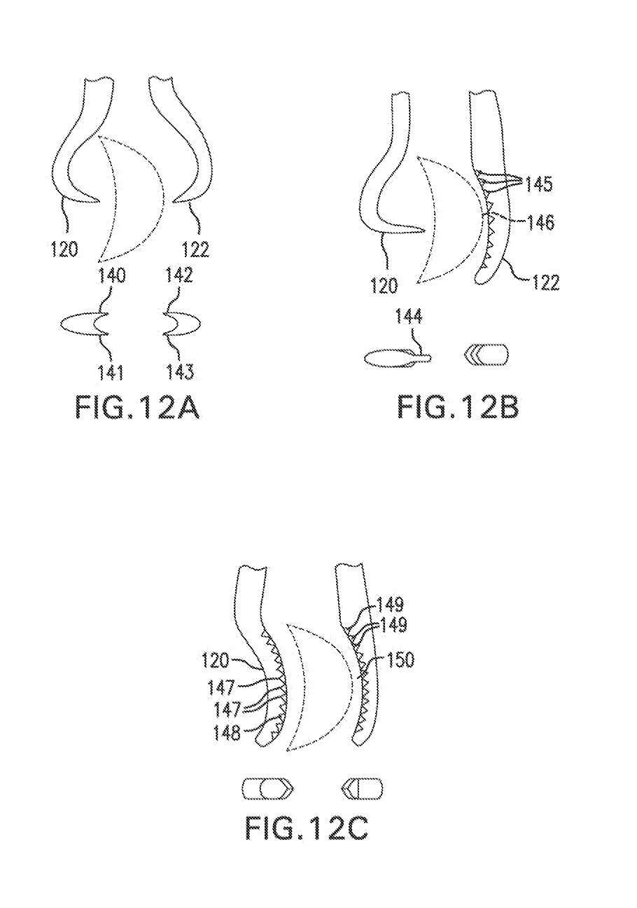

Various embodiments of medical devices 100 of the disclosed subject matter are provided in FIGS. 12A to 12C. FIG. 12A depicts medical device 100 including first engaging member 120 having first point or tooth 140 and second point or tooth 141, and second engaging member 122 having third point or tooth 142 and fourth point or tooth 143. In another embodiment as illustrated in FIG. 12B, the first engaging member 120 has a single point 144 and the second engaging member 122 has a series of serrated teeth 145 along a concave surface of a curved portion 146. In yet another embodiment as illustrated in FIG. 12C, the first engaging member 120 has a series of serrated teeth 147 along a convex surface of a curved portion 148 and the second engaging member 122 a series of serrated teeth 149 along concave surface of a curved portion 150.

Table 1 illustrates the unexpected superior results a medical device 100 having a single point to provide the greatest “bite” with minimal damage to bone, serrated teeth to provide the superior stability against twisting, and sufficient curvature to pass around an obstruction, such as a dorsal lip, to reach distal surfaces of a bone, as shown in FIGS. 12A, 12B, and 12C.

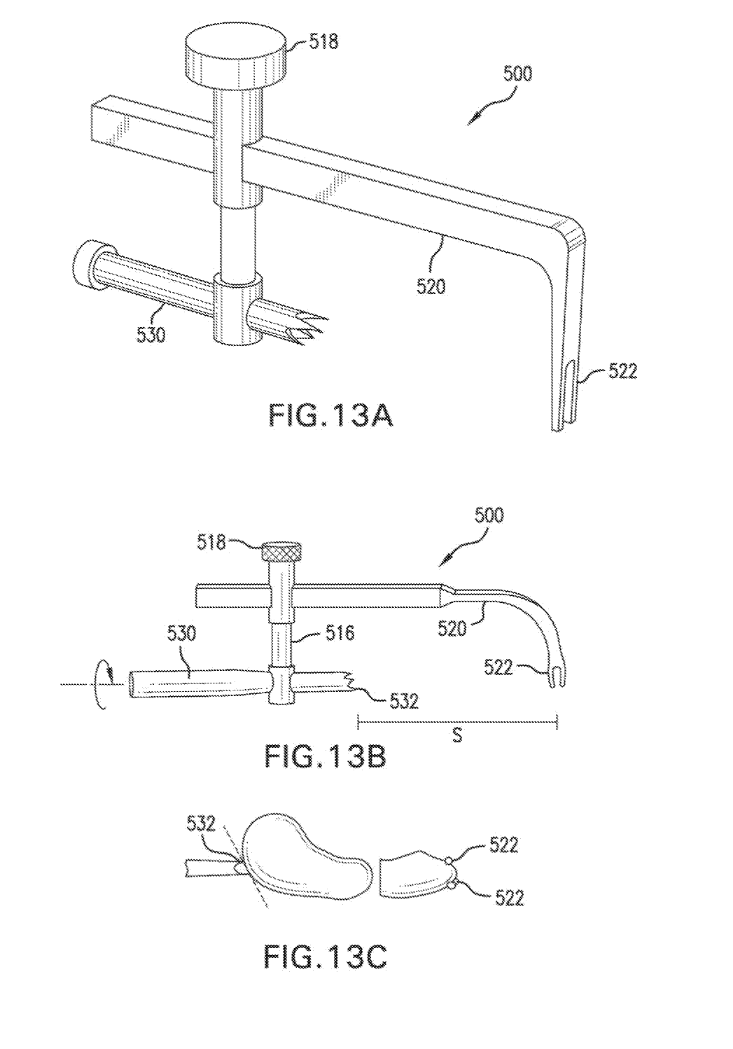

In another aspect, a medical apparatus 500 or jig is provided, as shown in FIGS. 13A and 13B. The medical apparatus 500 or jig aids the proper positioning of an implant to maintain alignment of the reduced bones, such as the scaphoid and lunate.

Referring to FIG. 13A, medical apparatus 500 provides an improved device for inserting an implant into fractured bone segments. The medical apparatus 500 generally includes a barrel 530 and an extendable member 520 connected by body 516, e.g., shaft.

In one embodiment, the barrel is rotatable as shown in FIG. 13B. The barrel can further include at least one end that is angled. It has been found that the angled barrel better fits the anatomy of the scaphoid bone. The rotation of the barrel 530 allows a better fit to the variable anatomy of the bones to be reduced. In some embodiments, the rotatable barrel 530 includes at one end an incisive surface 532 along an end of the barrel body 530. The incisive surface 532 stabilizes engagement of the barrel 530 to the bone. The incisive surface 532 for example, as shown in FIGS. 13A and 13B can include a plurality of teeth. The barrel 520 is rotatable with respect to the shaft 516 and the barrel end or tip 532 is angled in order to ensure a high conformance with the bone, e.g., the surface of the right wrist's scaphoid or left wrist's scaphoid at the scaphoid step-off angle.

It has been found that prior art jigs, such as the Huene jig is not well-suited for bones that have a personal or significant curvature. For example, the Huene jig and other available jigs are only designed to work on surfaces perpendicular to their major axis and not those that are oriented obliquely. Thus, the jigs of the prior art are not optimal for bones such as the scaphoid, which has a variable curvature. In other words, different people have different degrees of curvature, thus, the one size fits all jigs that are available in the art cannot compensate for the differences in bone structure across a population. Medical apparatus 500 has a rotatable barrel which can be useful to accommodate the scaphoid and lunate bones having different structures and morphologies.

The extendable member 520 includes a targeting member 522 that is configured to attach onto the bone in a stable manner. In one embodiment, the targeting member 522 is bifurcated into two bone-contacting points. The bifurcated targeting member 522 at a distal end of the extendable member 520 provides improved stability over prior art devices, in particular for the lunate bone and other bones that have a morphology with a high degree of curvature at the tip, i.e., generally pointed. The curved or pointed bone can be well secured between the two points of contact in the bifurcation. However, other configurations may be employed depending on the bone to be targeted.

Shaft 516 interconnects the barrel 530 and extendable member 520. Shaft further includes an actuator 518, such as a singular tightening mechanism, that can simultaneously allow control of rotation of the barrel 520 and extension of the targeting member 520. Thus, extendable length of travel (“S”) of the extendable member enables the medical apparatus to span both the scaphoid and lunate bones rather than just the scaphoid bone as prior art devices, such as that described in U.S. Pat. No. 5,312,412, and is herein incorporated by reference for all purposes. Medical apparatus 500 is an improvement over the prior art for RASL procedures and other procedures that require association of bones, and in particular, bones with curved or irregular surfaces.

In practice, apparatus 500 is used to ensure that the cannulated screw or implant is placed in the correct position within the scaphoid and lunate. The spacing S between the targeting member 522 and the barrel tip 532 is adjustable, e.g., by sliding the extendable member 520 and/or barrel 530 relative to the shaft 516. Apparatus 500 is adjusted so that barrel tip 532 is brought into contact with the scaphoid and the targeting member 522 is brought into contact with the lunate, as illustrated in FIG. 13B. Controller 518 is used to lock extendable member 520 and barrel 530 with respect to shaft 516. A guide wire can be inserted through the bore of a hollow rotatable barrel 530 and into the scaphoid and the lunate. Then, a pilot hole for an implant can be drilled. After the pilot hole is drilled, the implant may be inserted into the bones to maintain proper alignment and fixation postoperatively. Medical apparatus 500 provides precise, reproducible placement of the implant, which may reduce the incidence of complications and revisions.

Referring to FIG. 14, in an alternative embodiment, various aspects of medical device 100 and medical apparatus 500 may be combined into a combination article 200 including a post-reduction clamp 220 that holds both the scaphoid and lunate in place so that any K-wires that may be used can be removed. Upon satisfactory reduction, either with prior art methods utilizing K-wires or with methods using medical device 100, combination article 200 may be used to hold the reduction in place. At this point, K-wires or medical device 100 may be removed from the bone. In one embodiment, the combination article 200 incorporates a curvature to the clamp that allows it to fit the curvatures formed by the combined distal and proximal joint surfaces of the scaphoid and lunate (the so-called “carpal arcs”). The combination article incorporates a reversible drill guide specifically designed so that the guide wire can be positioned directly in the center of the scaphoid and lunate, whether from the left in the case of a right wrist or vice versa. Placement of the guide wire, and eventually the implant, in an axis that coincides with the center of the scaphoid and lunate, closely approximates the ideal axis for the implant. This guide can be designed to define the beginning and/or endpoints of the drill bit for drilling the pilot hole, and thereby the beginning and endpoints of the screw, or it can be designed to provide the surgeon with the ability to make precise corrections to the angle and position of the guide wire, drill bit, and screw along all three axes. At the proximal end of the device a handle 240 for opening and closing the clamp 220 is provided. The combination article can further include a guide 210 to provide for accurate placement of the implant. The drill guide specifically designed so that the guide wire can be positioned above the scaphoid and lunate, and adjusted via fluoroscopic imaging to be in the proper axis, may be attached to the clamp. Ideally, this guide should be left and right reversible.

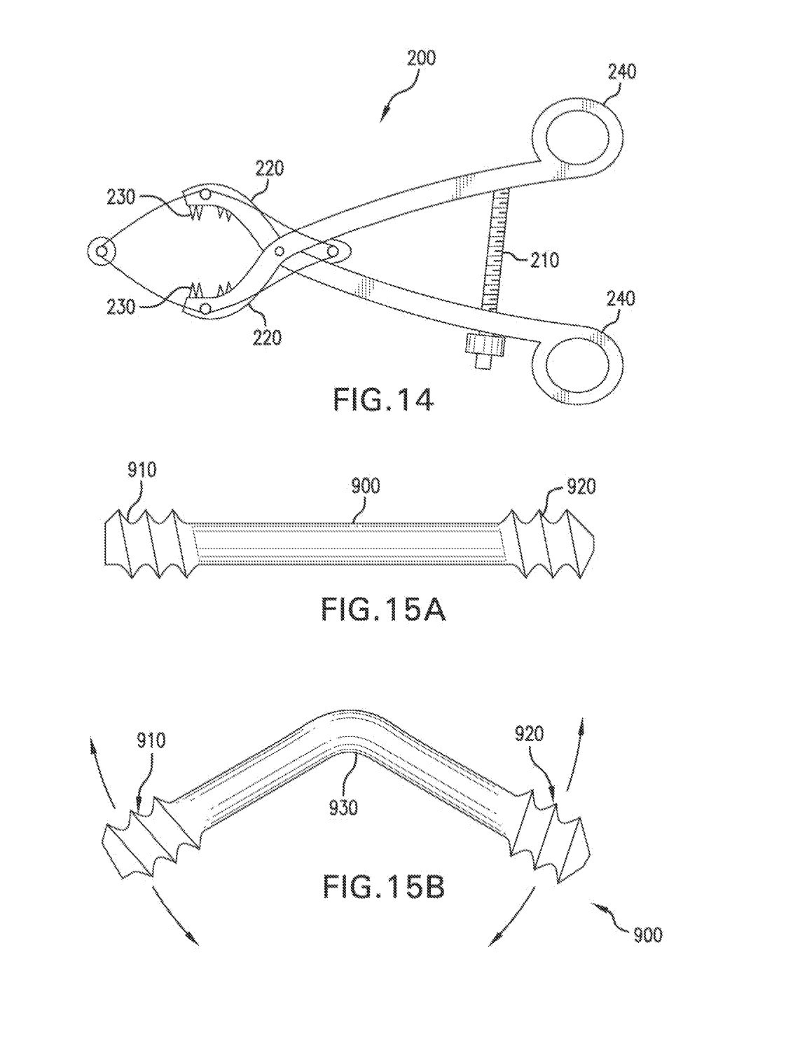

In yet another aspect, an implant for maintaining alignment of reduced bones is provided. Referring to FIGS. 15A and 15B implant 900 generally includes a bendable shaft 930, as best viewed from FIG. 15B. The implant allows for increased physiologic motion post-operatively between the bones as compared to a rigid implant. The implant includes a first end 910 a second end 920 and an intermediate portion 930. The first end 910 and second end 920 are rigid. The intermediate portion 930 is fabricated from a flexible material to allow for axial motion. For example, an angle defined at intermediate section 930 by the bending of first and second ends 910 and 920 can be about 15 to 20 degrees. Existing screws are not bendable and if not implanted at the perfect angle, the implanted screw pushes against bone as the subject moves the joint, such as the wrist with implanted screw. This sometimes causes screws to break in vivo, or damage to the bone. The implant 900 described herein has sufficient degrees of bend to allow an implant that may not have been inserted at the perfect axis to have a bit of “give” so that the bone does not become damaged. In one embodiment, the amount of bend is about a 15 to 20 degree angle or an angle that is sufficient to cover the spectrum of movement that could be encountered at the joint naturally. Such flexion at the intermediate portion of the implant provides a surgeon with a larger margin for error in placing the implant with its axis aligned with the axis of the instant center of motion of the two bones. The implant could be adapted for the scaphoid and lunates bones, or larger joints, e.g., knee, elbow, ankle, to replace or supplement damaged ligaments in those joints as well. In particular, the implant can be manufactured in various sizes depending on the indication. For the purpose of illustration and not limitation, the implant can have a length of about 2.5 mm to about 60 mm, and exemplary diameters are between 2.0 mm to about 10.0 mm, depending on the indication.

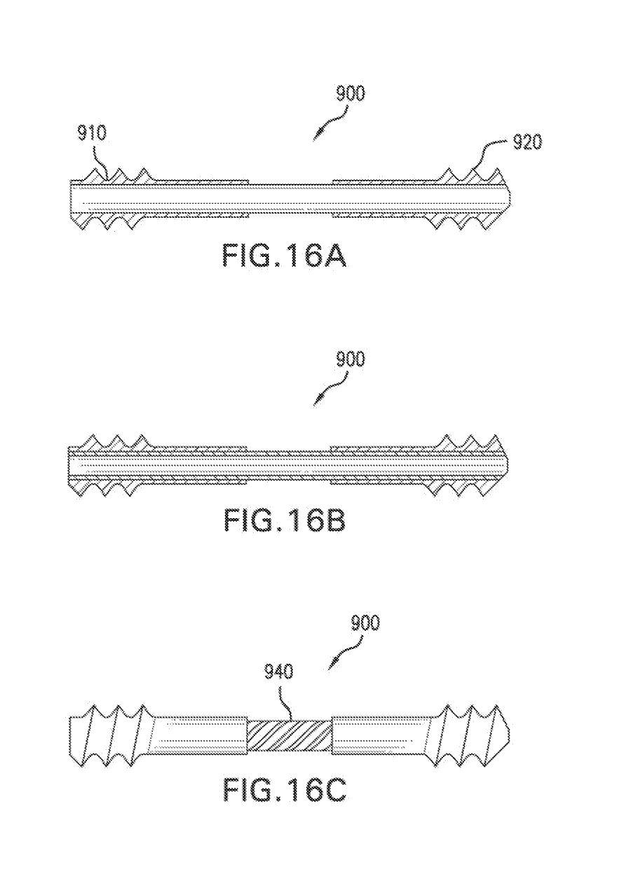

Implant 900 may be a cannulated screw that is formed, at least in part from a nickel titanium alloy, e.g., Nitinol, such as for example, those shown in FIGS. 16 A to C and 17 A to C. The intermediate portion 930 may be comprised of a Nitinol member 940 (e.g., Nitinol mesh, stent, or wire) that is integrated into a cannulated implant 930. In this manner, the cannulated implant 900 may be cut in half and a nitinol member, such as a nitinol mesh or stent-like structure can be press-fitted into the two halves of the implant and rejoined. In one embodiment, the Nitinol member is inserted into the implant after the two halves are inserted into bone. In one embodiment, the Nitinol member can be composed such that at room temperature, the Nitinol member would be in a martensitic state, whereas at body temperature the structure becomes austenitic allowing for expansion. Expansion of the structure after it is inserted into the deployed implant joins the halves to form a headless screw having a flexible intermediate.

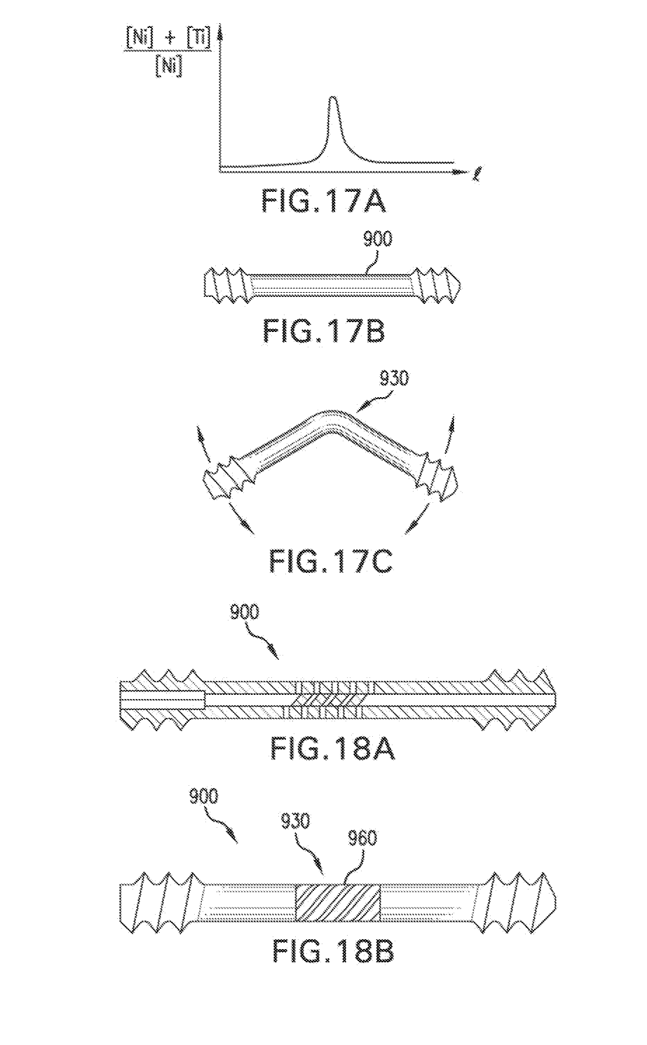

Alternatively, the implant may be manufactured from a superelastic alloy, such as Nitinol, as shown in FIGS. 17B and 17C. In this embodiment, the Nitinol alloy may include a varied concentration of nickel along the length of the implant shaft, such as shown in FIG. 17A. As depicted, the terminal ends of the implant include less nickel than the intermediate portion of the implant. Accordingly, the implant will include a bendable intermediate portion due to the change in concentration of nickel, as shown in FIG. 17C.

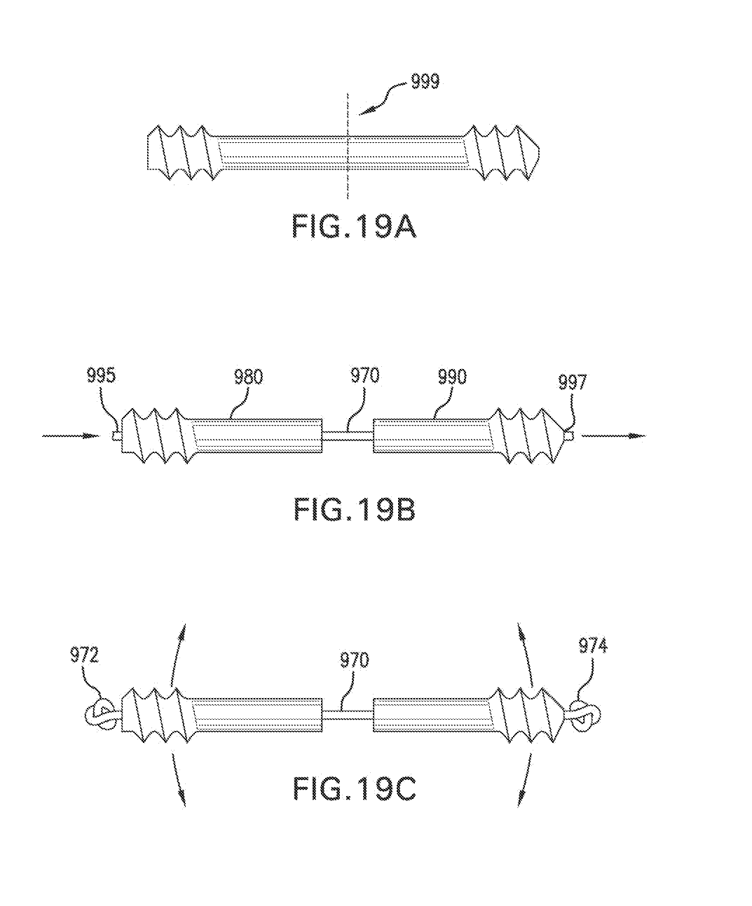

In another embodiment, implant 900 can comprise a hollow metal tubular member having an intermediate section with a plurality of cuts 960 along a length thereof. The cuts, for example, as shown in FIG. 18A can extend through the wall of the tubular member, thereby making the intermediate section having a greater flexibility than the sections distal and proximal to the intermediate section.

In yet another embodiment, implant 900 can include an intermediate section 930 formed from a wire 970, as depicted in FIGS. 19B and 19C. In this regard, the implant can comprise first and second tubular sections 980 and 990 proximate to the wire section 970. As schematically shown a tubular implant 900 can be cut in half (FIG. 19A), and a wire can be inserted into the tubular members 980 and 990. The wire 970 can extend through the opposing ends (995, 997) of the implant and the opposing ends of the wire 970 be knotted (972, 974) to securely fasten the wire to the implant 900.

In another embodiment, the implant 900 can be formed at least in part from polymeric or natural biomaterials. In this embodiment, the entire implant or the intermediate section can be formed from the biomaterial. The biomaterial can serve as a scaffold to foster ligament neogenesis for biological healing. The biomaterial may additionally incorporate growth factor for delivery to the site. In one embodiment, the biomaterial comprises polymeric fibers of polylactide-co-glycolide, for example, in a 10:90 ratio. The biomaterial can be fabricated using three-dimensional braiding technology. In another embodiment, the biomaterial can comprise collagen, such as collagen type I fiber-based scaffolds. A braid-twist scaffold design can be employed, and scaffold can be left uncrosslinked or crosslinked after the addition of gelatin, or crosslinked without gelatin.

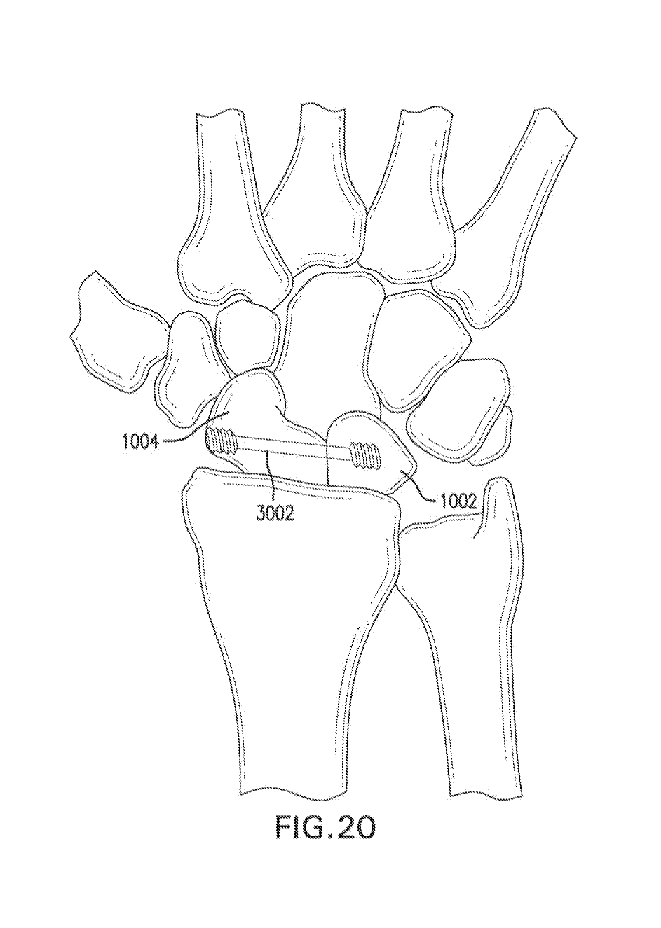

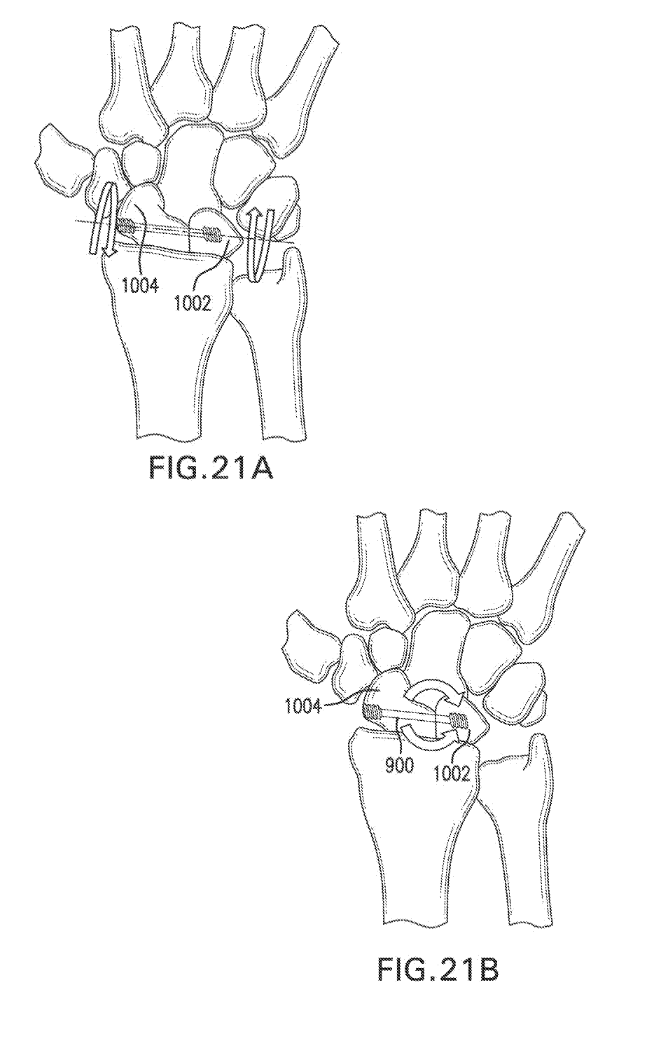

The implant 900 can allow for elastic deformation in the intermediate portion 930 which can reduce the incidence of complications and allow subject to regain motion that is closer to their physiological baseline levels. Further, by allowing the implant to bend or flex in multiple directions without significantly compromising the strength in tension, the implant effectively acts as an artificial ligament in between two bones. The threads remain firmly locked in the two adjacent bones, with the majority of the motion and stresses in the joint being accepted by the flexible, central portion of the screw. The threaded portions of the screw are made of titanium, and threaded in a conventional manner. Currently, no implant allows for the implant to bend or flex in multiple different directions. For example, FIG. 20 scaphoid lunate fixation with a cannulated screw 3002 of the prior art. As shown in FIG. 21A, the allowed axis of motion with use of current implants (shown in FIG. 20) is limited to rotation and de-rotation about a longitudinal axis of the screw. In contrast, as shown in FIG. 21B, implant 900 provides toggle (rotation in other planes) that is currently impossible with existing technology.

In some embodiments, the central portion of the implant uses a nickel-titanium alloy to imbue the shaft with controllable superelastic properties that can withstand substantial deformation and cyclical load without failure. Nitinol has been developed substantially for use in the body for a number of applications, with coatings and formulations developed to minimize deleterious effects on the body through the release of small particles and oxidative byproducts. The nickel titanium intermediate portion takes the form of, e.g.: (a) a braided strand, similar to Nitinol cardiac stents (or actual cardiac stents adapted for this purpose), press-fit or otherwise mechanically integrated into traditional titanium threads; (b) a smooth, single implant with a nickel concentration that varies across the length of the shaft, with the nickel concentration being greatest in the intermediate region and dropping off to zero in the threaded portions; or (c) a combination of the two. While some embodiments employ a nickel titanium alloy as the flexible material, other materials may be preferable, such as threaded isoelastic polymer cables.

While the flexible implant would be most immediately useful for the RASL procedure, small adjustments to the scale of the implant would allow it to be adapted for the replacement of ligaments across most any joint, including the knee, elbow and ankle, among others. The device, apparatus, and implant described herein will fill a clear void in the treatment of ligament injuries that spans the gap left between mechanical solutions that limit motion or are prone to failure, and biological and bioengineered solutions that are lacking in mechanical strength or viability.

In accordance with another aspect, a modular kit is provided that includes medical device 100, medical apparatus 500 and implant 900. In one embodiment, the modular kit is a RASL kit, which includes the system having implant 900 configured for the scaphoid and lunate bones, e.g., suitable length and diameter. In another embodiment, for example, the modular kit can be for the ankle or knee. With respect to such subject matter, the modular components would be included in sizes that are well-suited for the particular indication, e.g., calcaneal, or forefoot-midfoot indications, etc.

In yet another aspect, a reduction tool is provided to precisely facilitate the rotation and association of the fractured bones. Referring to FIG. 22, reduction tool 1300 includes a first arm 1310 and second arm 1320 each including first and second connectors (1340, 1350) configured to receive and hold first and second medical devices 100 and 100′. The reduction tool further includes a dial-up member 1330 that connects the first and second arms and facilitates incremental movement of the first medical device 100 and second medical device 100′.

First and second connectors 1340 and 1350 are slidingly engaged to the first arm 1310 and second arm 1320, respectively. In one embodiment, dial up member 1330 includes a semicircular member having a radius of curvature comprising an arc. The dial-up member can measure the angle and degrees of rotation of the fragmented bones.

The reduction tool 1300 allows the surgeon to measure the degree of rotation or de-rotation necessary to properly re-align the fragmented bones to their correct positions and position the first and second medical devices 100, 100′ along the length of first and second arms 1310, 1320 such that the bones grasped by the first and second medical devices can be rotated to the precise degree of rotation for proper positioning.

The incremental movement of the first and second medical devices allows the user to move the medical devices 100, 100′ to reduce the fragmented bones, using precise angular measurements obtained via pre-operative radiographs that allow for assessment of the degree of dissociation. The reduction tool allows for precise reduction of toggle in the transverse plane via member 1330, reduction of malrotation in the sagittal plane via the precise placement of connectors 1340 and 1350 along the arms 1310 and 1320, and reduction of toggle in the coronal plane via rotation of the medical devices 100 and 100′ within the connectors 1340 and 1350. Each rotation can be pre-determined ahead of time, either preoperatively or intra-operatively, and the exact amount of angular displacement necessary to achieve reduction dialed-in as such. The arcs formed by members 1330, 1310, and 1320 have equal radii of curvature, such that they all converge at a common center, which allows for precise control of translation to ensure that the bones do not become displaced linearly with respect to one another. For example, the RASL procedure requires precise alignment across 6 degrees of freedom (relative rotation in the sagittal plane, toggle in the coronal and transverse planes, and translation in the above-mentioned planes). No current technology is available to perform such dial up reduction for the precise measurement of reduction and association of the fragmented bones. In some embodiments, the reduction tool is made of a radiolucent material so that radiographs may continue to be used during the procedure to assess reduction and fine-tune as necessary. In some embodiments, the entire reduction tool or components thereof are transparent in order to minimize the direct obstruction of the surgeon's view.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

The foregoing description of specific embodiments of the disclosed subject matter has been presented for purposes of illustration and description. It is not intended to be exhaustive or to limit the disclosed subject matter to those embodiments disclosed. The disclosed subject matter is also directed to other embodiments having other possible combinations of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations.

What is claimed is:

1. A medical device, comprising:
a body having a first end and a second end,
a first engaging member positioned adjacent the first end of the body and adapted to operatively engage a carpal bone, and
a second engaging member positioned adjacent the first end of the body and adapted to operatively engage the carpal bone
wherein the position of at least one engaging member is adjustable with respect to the body.

2. The medical device of claim 1, wherein at least one of the first engaging member and the second engaging member includes at least two bone-contacting points.

3. The medical device of claim 1, wherein the first engaging member includes at least two bone-contacting points and the second engaging member includes at least two bone-contacting points.

4. The medical device of claim 1, wherein the first engaging member includes a curved toothed bone-contacting surface and the second engaging member includes a single bone-contacting point.

5. The medical device of claim 1, wherein the first engaging member includes a curved toothed bone-contacting surface and the second engaging member includes a curved toothed bone-contacting surface.

6. A medical apparatus, comprising:
a barrel including a longitudinal body having an angled first end adapted to engage a first portion of a bone,
a targeting member having a longitudinal body having a first end adapted to grip a second portion of the bone,
a shaft disposed between and connecting the barrel and the targeting member, wherein an adjustable space is defined between the angled first end of the barrel and the first end of the targeting member.

7. The medical apparatus of claim 6, wherein the barrel is hollow.

8. The medical apparatus of claim 6, wherein the barrel has a rotatable body.

9. The medical apparatus of claim 6, wherein the targeting member has a bifurcated end.

10. The medical apparatus of claim 6, wherein the angled end of the barrel includes an incisive surface.

11. The medical apparatus of claim 6, wherein the targeting member is slidably engaged to the shaft.

12. The medical apparatus of claim 6, wherein the space defined between the first end of the barrel and the first end of the targeting member is adjustable by longitudinal movement of at least one of the targeting member or the barrel.

13. A medical implant comprising,
a longitudinal body including
a first end portion,
a second end portion, and
a bendable intermediate portion disposed between the first and second ends.

14. The medical implant of claim 10, wherein the first end portion and the second end portion are rigid.

15. The medical implant of claim 10, wherein the first end portion and second end portion are threaded.

16. The medical implant of claim 10, wherein the intermediate portion allows bending but maintains its strength in tension.

17. The medical implant of claim 10, wherein the intermediate portion is made of Nitinol.

18. The medical implant of claim 10, wherein the implant is made of a nickel titanium alloy and wherein the concentration of nickel is greatest in the intermediate portion and least at the end portions.

19. The medical implant of claim 10, wherein the first end portion is adapted to be anchored to a first bone and the second portion is adapted to be anchored to a second bone.

20. The medical implant of claim 16, wherein the first bone is a scaphoid bone and the second bone is a lunate bone.

21. A medical device comprising:
a longitudinal body having a first end and a second end,
first and second members configured to grip a bone, the first and second members each moveable from a first position to a second position,
an actuator operatively connected to the first and second members, the actuator configured to move at least one of the first or second members from the first position to the second position.

22. The medical device of claim 18, wherein the first and second members include one or more points configured to penetrate bone.

23. The medical device of claim 18, wherein the actuator includes a mechanism to incrementally move the at least one of the first and second members between a first position and a final position.

24. A medical tool for reducing first and second bones, the medical tool comprising:
a first arm and a second arm, the first and second arms having a curvilinear body,
a third arm engaged to both the first and second arms, the third arm adapted to measure an angle of rotation of the first and second bones, the first and second arms are configured to receive a first medical device and a second medical device adapted to grip first and second bones.

25. The medical tool for reducing first and second bones of claim 24, further including first and second connectors engaged to the first and second arms, the first and second connectors adapted to receive first and second medical devices.

26. The medical tool for reducing first and second bones of claim 24, wherein the third arm is a dial-up member adapted to measure the angle or degrees of rotation of first and second bones.

27. The medical tool for reducing first and second bones of claim 26, wherein the first and second medical devices can be rotated in response to the measured angle or degrees of rotation of the first and second bones to realign the first and second bones.
